(19) United States
(12) Patent Application Publication
(10) Pub. No.: US 2017/0246440 A1
(43) Pub. Date: Aug. 31, 2017

(54) APPARATUSES AND METHODS FOR PLASMAPORATION USING MICRONEEDLES

(71) Applicant: EP Technologies LLC, Akron, OH (US)

(72) Inventors: Sameer Kalghatgi, Copley, OH (US); Abhishek Juluri, Akron, OH (US); Tsung-Chan Tsai, Worthington, OH (US); Robert L. Gray, Kent, OH (US)

(21) Appl. No.: 15/442,824

(22) Filed: Feb. 27, 2017

Related U.S. Application Data
(60) Provisional application No. 62/300,976, filed on Feb. 29, 2016.

(51) Int. Cl. A61M 37/00; A61N 1/44

(52) U.S. Cl. CPC A61M 37/0015; A61N 1/44; A61M 2037/0023; A61M 2037/0061; A61M 2037/0046; A61M 2037/003; A61M 2037/0007; A61M 2205/3317

(57) ABSTRACT

Apparatuses and methods for delivering bioactive substances or cosmetic substances using plasmaporation and microneedles are provided. The delivery of the substances includes topical, intracellular, intercellular, and transdermal delivery to the subject.

### RELATED APPLICATIONS

This application claims the benefits of and priority to U.S. Provisional Application Ser. No. 62/300,976, titled APPARATUSES AND METHODS FOR PLASMAPORATION USING MICRONEEDLES, which was filed on Feb. 29, 2016 and is incorporated herein by reference in its entirety.

### FIELD

This disclosure relates to apparatuses and methods for delivering substances, such as, for example, bioactive substances and cosmetic substances, using plasmaporation devices that include microneedles.

### BACKGROUND

Transdermal delivery of drugs is an appealing alternative to oral or intravenous delivery of drugs. The administration of drugs through the skin can eliminate degradation in the gastrointestinal tract, an occurrence associated with oral delivery; as well as reduce the pain and inconvenience of intravenous injection. One of the most promising approaches of transdermal delivery of biotechnology-based drugs and other substances is the use of microneedles. Microneedles provide a minimally invasive means to deliver molecules into the skin by creating micron-sized holes to a depth of 50-200 μm in the stratum corneum without inducing pain. Because nerve endings are situated deeper than the depth to which the microneedles penetrate, there is no sensation of pain. While microneedles can deliver drugs through the skin, microneedles cannot deliver drugs into cells.

Various enhancement techniques can be used in combination with microneedles in order to further improve the delivery of the drug. Such techniques may include electroporation. Electroporation of tissues involves the application of one or more direct current high-voltage pulses of short duration. This application of high voltage electric pulses to tissue leads to cell membrane permeabilization (or in other words, opens pores) and electrophoresis of large charged molecules such as DNA.

However, electroporation requires electrode contact with skin or insertion in to skin, tissue or muscle and direct electric current application to promote cellular uptake of the drug. This direct electrode contact and direct current application to the skin has drawbacks including pain, electric shock, involuntary muscle contractions upon application, and can cause current induced tissue damage. These drawbacks have limited electroporation's widespread adoption for topical and transdermal drug delivery.

### SUMMARY

In accordance with the present disclosure, apparatuses and methods for delivering substances, such as bioactive substances and cosmetic substances, using plasmaporation devices that include microneedles are provided.

An exemplary apparatus includes a plasmaporation device comprising microneedles and a non-thermal plasma generator, wherein the non-thermal plasma generator generates a pulse having a voltage of about 1 kV to about 30 kV and a pulse duration ranging from about 1 ns to about 10,000 ns, and wherein, in some embodiments, the non-thermal plasma generator operates at a pulse frequency of about 1 Hz to about 30,000 Hz for up to about 180 s, and in some embodiments operates with a selected number of pulses, such as for example, from about 1 to about 100,000 pulses, and one or more electrodes. In some embodiments, the one or more electrodes may be one or more microneedles.

An exemplary method of delivering substances includes using a plasmaporation device on cells or tissue of a target area of a subject, wherein the plasmaporation device includes hollow microneedles, a non-thermal plasma applicator (which includes a generator and at least one electrode), and a reservoir containing a substance in fluid communication with the hollow microneedles; providing power to generate a plasma by energizing the non-thermal plasma electrode with a pulse having a voltage of about 1 kV to about 30 kV and a pulse duration ranging from about 1 ns to about 10,000 ns. In some embodiments the non-thermal plasma generator operates at a pulse frequency of about 1 Hz to about 30,000 Hz for up to about 180 s, and in some embodiments, it operates with a selected number of pulses, such as for example, from about 1 to about 100,000 pulses. The generated plasma porates the cells or tissue of the target area. The method also includes delivering a substance from the reservoir through the hollow microneedles into the porated cells or tissue of the target area of the subject. Further in accordance with this exemplary method, delivering the substance from the reservoir includes contacting the microneedles to the target area of the subject. In some embodiments, the microneedles are the electrodes.

An exemplary method of delivering substances includes using a plasmaporation device on cells or tissue of a target area of a subject, wherein the plasmaporation device includes hollow microneedles, a non-thermal plasma applicator (which includes a generator and at least one electrode), and a reservoir containing a substance in fluid communication with the hollow microneedles; delivering the substance from the reservoir through the hollow microneedles into the cells or tissue of the target area of the subject; and providing power to generate a plasma by energizing the electrode with a pulse having a voltage of about 1 kV to about 30 kV and a pulse duration ranging from about 1 ns to about 10,000 ns. In some embodiments, the non-thermal plasma generator operates at a pulse frequency of about 1 Hz to about 30,000 Hz for up to about 180 s, and in some embodiments, it operates with a selected number of pulses such as from about 1 to about 100,000 pulses. The plasma porates the cells or tissue of the target area to facilitate transfer of the substance into the porated cells or tissue. Further in accordance with this exemplary method, delivering the substance from the reservoir includes contacting the microneedles to the target area of the subject. In some embodiments, the microneedles are also the electrode.

An exemplary method of delivering substances includes using a plasmaporation device on cells or tissue of a target area of a subject, wherein the plasmaporation device includes solid microneedles and a non-thermal plasma generator, and wherein the solid microneedles are at least partially coated with a substance for delivery through the target area; delivering the substance by contacting the target area of the subject with the solid microneedles at least partially coated with the substance; generating a plasma by causing the non-thermal plasma generator to deliver one or more pulses having a voltage of about 1 kV to about 30 kV and a pulse duration ranging from about 1 ns to about 10,000 ns. In some embodiments, the non-thermal plasma applicator operates at a pulse frequency of about 1 Hz to about 30,000 Hz for up to about 180 s, and in some embodiments, it operates with a selected number of pulses such as from about 1 to about 100,000 pulses. In accordance with this exemplary method, the plasma porates the cells or tissue of the target area to facilitate transfer of the substance to the subject. Delivering the substance occurs before or after applying power to the plasmaporation device to generate the plasma. In some embodiments, the microneedles are also the electrodes.

An exemplary method of delivering substances includes using a plasmaporation device on cells or tissue of a target area of a subject, wherein the plasmaporation device includes solid or hollow microneedles and a non-thermal plasma generator; providing power to the plasmaporation device to generate a plasma to thereby create or modify pores by energizing the electrode by a pulse having a voltage of about 1 kV to about 30 kV and a pulse duration ranging from about 1 ns to about 10,000 ns. In some embodiments, the non-thermal plasma generator operates at a pulse frequency of about 1 Hz to about 30,000 Hz for up to about 180 s, and in some embodiments, it operates with a selected number of pulses such as from about 1 to about 100,000 pulses. The method also includes topically applying a substance to the cells or tissue of the target area of the subject containing the pores created or modified by the plasmaporation device. The substance is topically applied before or after the pores are created or modified. In some embodiments, the microneedles are the electrodes.

These and other features and advantages of the present invention will become better understood with regard to the following description and accompanying drawings.

### BRIEF DESCRIPTION OF THE DRAWINGS

FIGS. 1A1, 1A2 and 1B1, 1B2 are plan views and cross-sectional views of exemplary microneedle arrays 100A, 100B containing solid microneedles 103 used in accordance with the apparatuses and methods of the present disclosure.

FIG. 1C is a magnified elevational view of a microneedle 103 of the array 100B shown in FIG. 1B2.

FIGS. 1D1, 1D2 and 1E1, 1E2 each include a plan view and a cross-sectional view of exemplary microneedle arrays 150A, 150B containing hollow microneedles 153 used in accordance with the apparatuses and methods of the present disclosure.

FIG. 2 shows a cross-sectional view of an exemplary plasmaporation device 201 that generates a DBD plasma, the device 201 includes hollow microneedles 203 and encased electrodes 207 and a generator 211.

FIG. 3 shows a cross-sectional view of an exemplary plasmaporation device 301 that generates a DBD plasma, the device 301 includes solid microneedles 303 encasing an electrode 307.

FIG. 4 shows a cross-sectional view of an exemplary plasmaporation device 401 that generates a corona discharge plasma, the device 401 containing solid microneedles 403 comprising a conductive material and a generator 411.

FIG. 5 shows a cross-sectional view of an exemplary plasmaporation device 501 containing hollow microneedles 503, electrodes 507 and a plasma generator 511 for generating DBD plasma jets (“J”).

FIG. 6 shows a cross-sectional view of an exemplary plasmaporation device 601 containing hollow microneedles 603, electrodes 607 and a plasma generator 611 for generating DBD plasma jets (“J”).

FIG. 7 shows a cross-sectional elevation view of a portion of an exemplary plasmaporation device 701 in contact with and penetrating the epidermis of a subject.

FIG. 8 shows an elevational view of a portion of an exemplary plasmaporation device 801 having solid microneedles 803 encased in a grounded metallic mesh electrode 810.

FIG. 9 shows a cross-sectional elevation view of an exemplary plasmaporation device 901 having solid microneedles 903, electrodes 907 spaced from the target area 920 of the subject using a spacer component 912.

### DETAILED DESCRIPTION

Unless otherwise indicated herein, the term “bioactive substance” refers to antifungals, antimicrobials, opioids, growth factors, polynucleotides, oligonucleotides, peptides, RNAs, DNA plasmids, DNA-vaccines, RNA based vaccines, protein based vaccines, nanoparticles, liposomes, micelles, vesicles, quantum dots, cytokines, chemokines, antibodies, drugs such as, for example, non-steroidal anti-inflammatory drugs (NSAID's), biologics, such as, for example monoclonal antibodies or other proteins and peptides, and the like that may be delivered intercellularly, intracellularly, or both intercellularly and intracellularly via the plasmaporation devices disclosed herein.

Unless otherwise indicated herein, “circuit communication” refers to a communicative relationship between devices. Direct electrical, electromagnetic and optical connections and indirect electrical, electromagnetic and optical connections are examples of circuit communication. Two devices are in circuit communication if a signal from one is received by the other, regardless of whether the signal is modified by some other device. For example, two devices separated by one or more of the following—amplifiers, filters, transformers, optoisolators, digital or analog buffers, analog integrators, other electronic circuitry, fiber optic transceivers or satellites—are in circuit communication if a signal from one is communicated to the other, even though the signal is modified by the intermediate device(s). As another example, an electromagnetic sensor is in circuit communication with a signal if it receives electromagnetic radiation from the signal. As a final example, two devices are not directly connected to each other, but both capable of interfacing with a third device, such as, for example, a CPU, are in circuit communication.

Unless otherwise indicated herein, the terms “porate” or “poration” or other variations thereof refer to the act of creating new pores or more generally to the creation of new pores and/or the modification (e.g., enlargement) of existing pores.

Unless otherwise indicated herein, the term “tissue” refers to at least one of skin, epithelial tissue, mucosal tissue, connective tissue, muscle tissue, and nervous tissue.

The present disclosure is directed to moving substances, such as molecules, drugs, vaccines, or cosmetic substances, intercellularly, i.e., through the tissue, intracellularly, i.e., in to the cells, or both intercellularly and intracellularly, using microneedles and non-thermal plasma. In particular, the present disclosure is directed to moving such substances using a plasmaporation device comprising microneedles and a non-thermal plasma applicator.

In accordance with the present disclosure, the plasmaporation device comprising microneedles and a non-thermal plasma applicator utilize plasmaporation to deliver the substance, such as a bioactive substance, cosmetic substance or the like. Microneedles are a known form of transdermal delivery system for molecules, drugs, vaccines and the like. However, microneedles alone cannot efficiently deliver molecules, drugs, and vaccines intracellularly (into cells) due to the large size of microneedle tips (e.g., about 4 μm to about 100 μm), which is on the order of size of the cells in the skin. If such microneedles are inserted into cells, there is a possibility of cell damage, rupture, or cell death, particularly with relatively large microneedles. It is believed that the delivery of drugs, vaccines, and other molecules via microneedles may be enhanced with plasmaporation, without the unpleasant, undesirable, and sometimes damaging, side effects associated with electroporation or by hypodermic needle injection.

Plasmaporation, as referred to herein, is the use of non-thermal plasma, the fourth state of matter, to open up temporary pores in cells or tissue of a subject. The temporary pores, in turn, can be used for the intercellular and/or intracellular delivery of substances, such as bioactive or cosmetic substances, via the microneedle portion of the plasmaporation device. Non-thermal plasma is a partially ionized gas generated at atmospheric pressure using a strong electric field. It is generated by the breakdown of air or other gases present between two electrodes under the application of sufficiently high voltage. The plasma opens up new and temporary pores in the tissue and within cells of the target area to promote intercellular and/or intracellular delivery and uptake of the substances, such as bioactive or cosmetic substances. In some embodiments, the plasma may further modify pores already present. In some embodiments, the temporary pores remain open for about 1 to about 10 minutes. In accordance with embodiments disclosed herein, it should be understood that pores are created or modified in two ways to facilitate intercellular and/or intracellular delivery of the substances: in accordance with certain embodiments, the microneedle part of the plasmaporation device creates pores when it is inserted into or otherwise contacts the target area, and the plasma generated by the plasmaporation device creates additional pores or modifies existing pores (including, in some embodiments, modifying some that may have already been created by the insertion of the microneedles). The pores created by the physical insertion of the microneedles may be larger than those created by plasmaporation. Accordingly, the pores created by the plasmaporation device disclosed herein may be a combination of pores created from the insertion of microneedles and the pores created and/or modified by plasma.

It should be understood that with plasmaporation, the electrodes need not be in contact with the cells or tissue, no hypodermic needles are required, and generation of non-thermal plasma directly on the cells or tissue is rapid and painless. Accordingly, the apparatuses and methods utilizing plasmaporation as described herein provide efficient intercellular, intracellular, or both intercellular and intracellular delivery and uptake of fluids, i.e., gases or liquids, comprising substances, such as bioactive substances or cosmetic substances. The devices and methods disclosed herein may be therapeutic devices for administering therapy on a body, such as a person's body, or non-therapeutic, such as for use in delivery of cosmetics. The delivery and uptake of the substances according to the apparatuses and methods of this disclosure are free of the pain, muscle contractions, and current induced tissue damage associated with electroporation, as well as the pain or discomfort associated with injecting hypodermic needles.

### Plasmaporation Device

As discussed above, the plasmaporation device of the present disclosure comprises microneedles and a non-thermal plasma applicator. In some embodiments, the plasma applicator includes the microneedles (if the microneedles are the electrode). In accordance with embodiments disclosed herein, the plasmaporation device is one of a dielectric barrier discharge (DBD) plasma generator, a DBD plasma jet plasma generator, and a corona discharge plasma generator.

### Microneedles

The plasmaporation devices of the present disclosure comprises a plurality of microneedles. The individual microneedles have a conical or frustoconical shape. In exemplary embodiments, the microneedles are arranged in arrays.

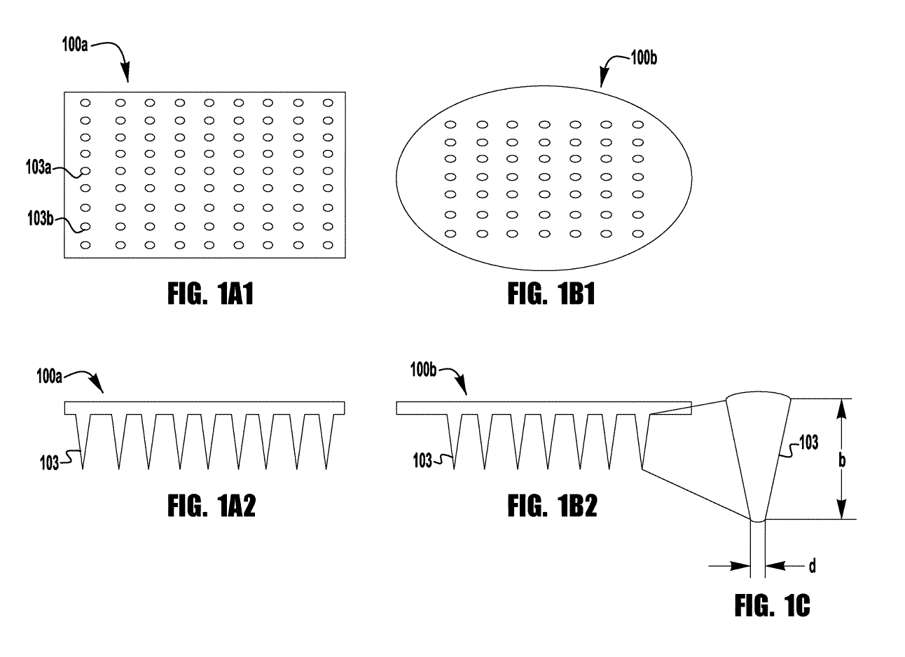

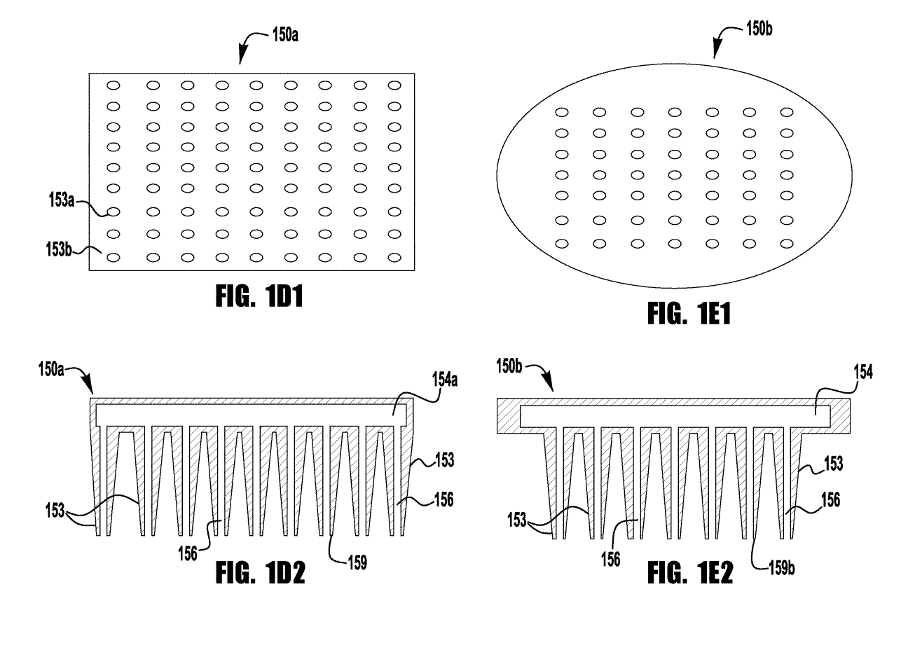

FIGS. 1A1-1E2 show exemplary microneedle arrays containing microneedles suitable as is, or with some modification (e.g. adding an electrode, coating, or the like) for use with the plasmaporation devices of the present disclosure. In exemplary embodiments, the microneedles 103, 153 are arranged in an arrays 100a, 100b, 150a, 150b. The plan view of FIGS. 1A, 1B, 1C, and 1D show how the individual microneedles (103 or 153) may be arranged within exemplary arrays 100a, 100b, 150a, 150b.

The microneedles in accordance with embodiments of the present disclosure may be solid microneedles (identified with a prefix of 103) or hollow microneedles (identified with a prefix of 153). In certain embodiments disclosed herein, all of the microneedles in the array are hollow microneedles. In other embodiments, all of the microneedles in the array are solid microneedles. In yet other embodiments, at least a portion of the microneedles in an array are hollow microneedles and at least a portion of the microneedles in the same array are solid microneedles. In certain of the preceding embodiments, at least a portion of the microneedles in the array is selected from solid microneedles, hollow microneedles, and combinations thereof.

As used herein, unless otherwise indicated, the term “solid microneedle” refers to a microneedle that does not contain a channel through which a fluid, such as a source gas for the plasma, a bioactive substance, or a cosmetic substance, can pass. FIGS. 1A and 1B show exemplary microneedle arrays 100a, 100b containing solid microneedles 103. FIG. 1A differs from FIG. 1B in the shape and array configuration only and thus, similar parts have like identifiers.

In contrast, as used herein, unless otherwise indicated, the term “hollow microneedle” refers to a microneedle that contains a channel through which a fluid, such as a source gas for the plasma, a bioactive substance, or a cosmetic substance, can pass. FIGS. 1D and 1E show exemplary microneedle arrays 150a, 150b containing hollow microneedles 153. The microneedles 153 may respectively have a channel 156 in fluid communication with a reservoir 154. FIG. 1D differs from FIG. 1E in the shape and array configuration only and thus, similar parts have like identifiers.

Channel 156 is configured such that a substance, e.g., a bioactive, cosmetic substance or the like, in gas, liquid, or both gas and liquid form, can pass through the microneedle 153. In certain embodiments of the present disclosure, the plasmaporation device comprises a reservoir 154 in fluid communication with at least a portion of the hollow microneedles 153 such that a substance may travel from the reservoir 154 through the hollow microneedle 153 and exit out of the tip 159 of the hollow microneedle 153.

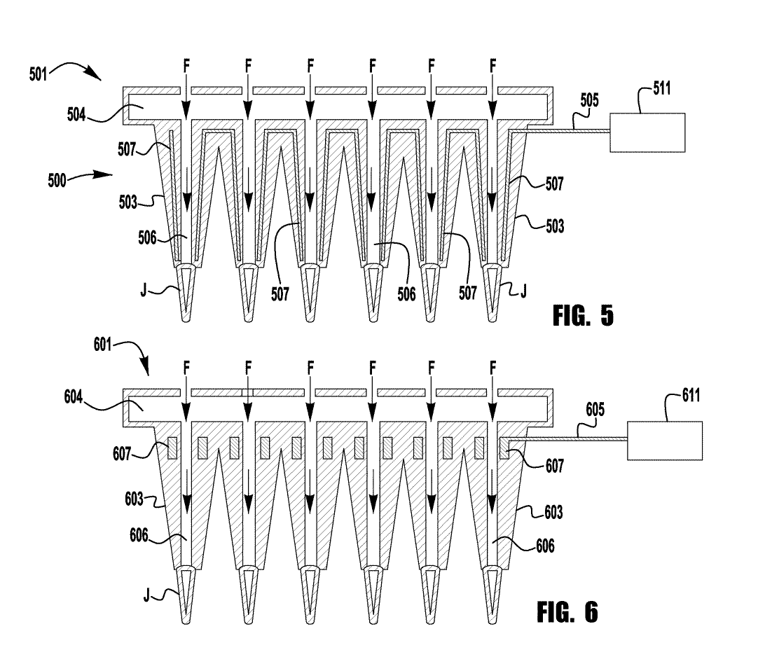

Referring to reservoir 154, in certain embodiments, the reservoir 154 is a sealed or is a sealable compartment capable of evacuating or driving a substance through the hollow microneedle 153. In some embodiments, the reservoir may be in fluid communication with a source gas intended to be driven through the hollow microneedles (for example, as shown by the flow “F” of fluid into reservoir 504 in FIG. 5). When a source gas is driven through the reservoir and/or microneedles, the substance in the reservoir and the source gas may be delivered through the hollow microneedle 153 in serial (one of the source gas or substance followed by another). Alternatively, when a source gas is driven through the reservoir, the source gas and substance may mix, and such mixture may be delivered through the hollow microneedle 153 as a mixture. In such embodiments, the plasma may interact with the substance being delivered, which may modify or completely change the nature of the substance being delivered, e.g., the plasma may oxidize the substance being delivered. In such embodiments, particularly when the bioactive substance is susceptible to modification by the plasma being generated, the substance is preferably protected from the plasma in some manner. In certain such embodiments for example, the substance may be encapsulated with a nanoparticle, liposome, or other protective material. In some embodiments, the fluid reservoir is configured to protect fluid contained therein from interacting with plasma generated by the plasma generator and electrode.

In certain embodiments, the reservoir further comprises a carrier fluid for a substance, such as a bioactive substance, a cosmetic substance or the like intended for the intercellular and/or intracellular delivery in a subject. The carrier fluid may be a gas that is the same or different than the source gas for the plasmaporation device. Non-limiting examples of suitable carrier fluids include noble gases, such as helium (He), argon (Ar), neon (Ne), xenon (Xe), and the like; molecular gases such as nitrogen (N₂); mixtures of any of He, Ar, Ne, Xe, and N₂ with molecular oxygen (O₂) where the oxygen comprises less than 1 wt % of the mixture (based on the total weight of the gas); and combinations thereof.

In accordance with the exemplary embodiments disclosed herein, the microneedles may have a height ranging from about 100 μm to about 2,000 μm. The height, unless otherwise indicated, refers to the distance “h” as shown in FIG. 1C, which represents the distance between the base and the tip of the microneedle 103. Further in accordance with exemplary embodiments of the present disclosure, the microneedles 103 may have a tip diameter ranging from about 4 μm to about 100 μm. The tip diameter, for example, refers to the length represented by “d” as shown in FIG. 1C. Unless otherwise indicated herein, the height “h” and diameter “d” parameters for a hollow microneedle 203 can be measured or determined in the same manner as the solid microneedle 103 shown in FIG. 1C.

The microneedles of the present disclosure may be further characterized by the configuration of the array. In general, the term “pitch” refers to the center-to-center distance between the microneedles in the array, e.g., the center-to-center distance between solid microneedles 103a and 103b shown in FIG. 1A, 1B or between hollow microneedles 153a and 153b shown in FIGS. 1D, 1E. In accordance with certain embodiments, the microneedles 103, 153 are in an array 100a, 100b, 150a, 150b having a pitch of about 10 μm to about 1000 μm, including from about 50 μm to about 1000 μm. The individual pitches between the microneedles 103, 153 in the array 100a, 100b, 150a, 150b may be the substantially the same or may vary throughout the array 100a, 100b, 150a, 150b so long as the individual pitches meet the aforementioned range, e.g., 10 μm to about 1000 μm. Unless otherwise indicated herein, the phrase “substantially the same” refers to dimensions or parameters that have minor differences, which may be due to manufacturing tolerances and processes, but otherwise have about the same intended design parameter. Typically, arrays that have substantially the same individual pitches have a regular periodicity, i.e., arrangement of rows and columns, of the microneedles in the array. Conversely, arrays that have varying individual pitches may have an uneven periodicity of the microneedles within the array, at least as compared to those having substantially the same individual pitches of the microneedles. In accordance with the embodiments disclosed herein, preferably, the arrays have substantially the same individual pitches between the microneedles in the array. In other words, in accordance with the embodiments disclosed herein, preferably, microneedle array has regular periodicity. In other embodiments, the microneedle array has an irregular periodicity.

Furthermore, in accordance with certain embodiments disclosed herein, the microneedles of the array have substantially the same shape as other microneedles within the array. The phrase “substantially the same shape” as used in this context refers to microneedles having the identical design parameters, i.e., the same design parameters for the height and/or the same design parameters for the lateral cross sections of the microneedles, but have minor differences in actual shape, which may be due to manufacturing tolerances and processes. Thus, in certain embodiments, all of the microneedles in the array are conical in shape. In other embodiments, all of the microneedles in the array are frustoconical in shape. In yet other embodiments, at least a portion of the microneedles in an array are conical in shape and at least a portion of the microneedles in the same array are frustoconical in shape. In certain of the preceding embodiments, the array of microneedles has microneedles having a conical shape, frustoconical shape, and combinations thereof.

In accordance with exemplary embodiments disclosed herein, the microneedles the array comprising the microneedles, or both may comprise a dielectric material. In accordance with certain exemplary embodiments disclosed herein, the microneedles the array comprising the microneedles, or both may comprise a conductive material. In accordance with exemplary embodiments disclosed herein, the microneedles the array comprising the microneedles, or both may comprise a dielectric material or a conductive material. In accordance with exemplary embodiments disclosed herein, the microneedles the array comprising the microneedles, or both may comprise a dielectric material and a conductive material. As mentioned above, the microneedles and non-thermal plasma applicator (which includes a generator and one or more electrodes (which may be the microneedles)) work together to produce the plasma used to create new temporary pores and/or modify existing pores in the cells or tissue of the subject.

The selection of the material for making the microneedle or microneedle array is a design criteria for the generation of the plasma. As discussed in greater detail below, when all or part of the exterior surface of the microneedle or microneedle array is a dielectric material, a dielectric-barrier discharge (DBD) plasma or, in certain embodiments for hollow microneedles a DBD plasma jet, may be generated by the plasmaporation device (i.e., the microneedles and the non-thermal plasma applicator, i.e. the generator and one or more electrodes) with a source gas in contact with, adjacent to, or proximal to, the dielectric material of the microneedle. In certain such embodiments that generate a DBD plasma or plasma jet, the dielectric material partially or fully encases or encloses a conductive material (electrode). When all or part of the microneedle or microneedle array includes a conductive material, a corona discharge plasma may be generated by the plasmaporation device (i.e., the microneedles and the non-thermal plasma applicator) with a source gas in contact with, adjacent to, or proximal to, the conductive material of the microneedle.

Unless otherwise indicated herein, the term “exterior surface” refers to a surface of the microneedles or microneedle array in contact with, or proximal to, a source gas for the plasma. Although the exterior surface is typically the external surface of the microneedles that contacts the skin, tissue or cells that is to be porated (e.g., the surface of the microneedle facing the target area of the subject), the term “exterior surface,” may also refer to the inner surface of hollow microneedles, namely the surface that forms channel in the hollow microneedles that will contact the fluid passing through the channel.

In accordance with embodiments disclosed herein, when the microneedles comprise a dielectric material, the dielectric material has a dielectric constant of about 10 to about 80. In certain embodiments, the dielectric material forms at least a portion of the exterior surface of the microneedles and/or microneedle array. Non-limiting examples of dielectric materials suitable for use in the microneedle or array include polytetrafluoroethylene (PTFE, commercially known as TEFLON), aluminum oxide (alumina), silicone, natural rubber, synthetic rubber, ceramic, polyetherimide (PEI, commercially known as ULTEM), quartz such as a dielectric quartz or fused silica, and magnesium fluoride.

In accordance with embodiments disclosed herein, when the microneedles and/or microneedle array comprises a conductive material; the conductive material has an electrical conductivity of about 10 Siemens/meter (S/m) to about 10⁸ S/m. Non-limiting examples of conductive materials suitable for use in the microneedle or array include metals such as stainless steel, gold, silver, copper, platinum, titanium, aluminum, indium tin oxide (ITO), and palladium; conductive polymers; and fullerenes such as carbon nanotubes and graphene.

Although some of the descriptions above refer to microneedles 103, 153 and arrays 100a, 100b, 150a, 150b, the descriptions, materials, arrangements, and the like are applicable to the other exemplary embodiments disclosed herein.

### Non-Thermal Plasma Applicator

As used herein, plasma applicator means a plasma generator and one or more electrodes. As discussed above, the plasmaporation device of the present disclosure comprises a non-thermal plasma generator in addition to the microneedles. The microneedles and non-thermal plasma applicator work together to generate DBD plasma, DBD plasma jets, or corona discharge plasma. The non-thermal plasma generator provides the electrical voltage used by the plasmaporation device to generate the plasma. As used herein, plasmaporation device includes the generator, the microneedles and one or more electrodes. In some embodiments, the microneedles are the electrodes. The non-thermal plasma applicator, which includes the generator, also may further provide the frequency and/or control for pulse duration and number of pulses used by the plasmaporation device to generate the plasma.

As mentioned above, non-thermal plasma is generated by the breakdown of a source gas (e.g., ambient air or other source gases disclosed herein) present between two electrodes under the application of sufficiently high voltage. In accordance with exemplary embodiments of the present disclosure, the microneedles and/or microneedle array may operate as one of the electrodes. Alternatively, an electrode may be embedded, encased, or otherwise covered by the microneedles and/or microneedle array.

When the microneedles and/or microneedle array operate as one of the electrodes, the non-thermal plasma applicator comprises at least one high voltage generator and the microneedles. For example, in certain embodiments, the microneedles and/or microneedle array comprise a conductive material in accordance with the conductive materials disclosed herein. In such embodiments, the microneedles and/or the array operate as an electrode. When the microneedles themselves and/or microneedle array operate as an electrode, the non-thermal plasma applicator comprising at least one high voltage generator is in circuit communication with the microneedles or microneedle array in such a manner that the plasmaporation device and the microneedles or microneedle array working together with the non-thermal plasma generator, generates a non-thermal plasma.

In accordance with certain embodiments of the present disclosure, when the microneedles and/or microneedle array is/are not an electrode and/or cannot operate as an electrode, the non-thermal plasma applicator further comprises at least one electrode. The microneedles and/or microneedle array may not be an electrode if it comprises a non-conductive material or a material that is not conductive as defined herein. In these embodiments, the at least one electrode is embedded, encased, proximate, or in contact with the microneedle and/or microneedle array such that when energized, the plasmaporation device generates a plasma.

In accordance with some embodiments of the present disclosure, the target area of the subject operates as the second electrode. For example, the at least one electrode in circuit communication with the non-thermal plasma generator is spaced apart from a second electrode, i.e., the target area of the subject, such that plasma is generated from the source gas (e.g., ambient air) in the gap between the microneedles and/or microneedle array and the target area of the subject. In certain embodiments, microneedles and/or microneedle array are connected to a spacer component (as shown in FIG. 9) in accordance with those described herein that provides a predetermined gap between the first electrode(s) and the second electrode, i.e., the target area of the subject. In some embodiments, the spacer component includes a grounding conductor to provide a ground path back to the power supply.

When the microneedle or microneedle array is a conductive material and operates as an electrode, the plasma generated is a corona discharge plasma. An exemplary plasmaporation device that would generate a corona discharge plasma is shown in FIG. 4, in which the array 400 and/or the microneedles 403 themselves function as at least one electrode in circuit communication with the high voltage generator 411 of the non-thermal plasma device 401 through high voltage conductor 405. It is contemplated that a corona discharge plasma plasmaporation device according to the present disclosure is operated such that it may contact the target area cells or tissue of the subject without adverse unpleasant effects (e.g., shocking the subject). One way to operate the plasmaporation device without adverse unpleasant effects is to, for example, limiting the pulse width of the applied voltage pulses.

In certain embodiments, the microneedle or microneedle array comprises a conductive material that functions as an electrode and is at least partially coated or covered by dielectric or other insulating material. The dielectric or insulating material creates a barrier between the electrode microneedles and/or microneedle array and the source gas used to generate the plasma (where the source gas is ambient air in contact with or proximate to the dielectric or insulated surface of the microneedles and/or microneedle array). The plasma generated by a plasmaporation device having this configuration (dielectric material at least partially coating or covering a microneedle/microneedle array electrode) is a DBD plasma or, in certain embodiments with hollow microneedles, a DBD plasma jet.

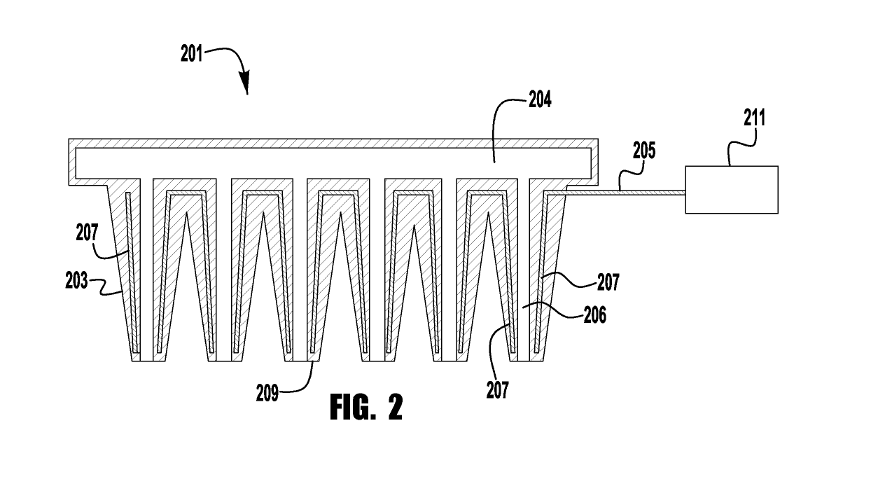

In some embodiments, the microneedles or the microneedle array is constructed or fabricated from a dielectric material as disclosed herein (as opposed to being coated by the dielectric material). In such embodiments, the plasmaporation device includes a non-thermal plasma applicator comprising at least one electrode and a generator. The at least one electrode is embedded, encased, adjacent to, or in contact with the microneedle and/or microneedle array such that when the non-thermal plasma applicator energizes, the plasmaporation device generates a plasma. As long as the least one electrode is embedded or encased such that the dielectric material of the microneedles and/or microneedle array forms a barrier between the at least one electrode and the source gas for the plasma, a DBD plasma or a DBD plasma jet is generated when energized. Exemplary plasmaporation devices comprising a DBD plasma generator may be represented by FIGS. 2, 3, and 5 in which the at least one electrode is in circuit communication (e.g., through a high voltage conductor) with the high voltage generator of the non-thermal plasma device and is embedded in dielectric microneedles or a dielectric microneedle array.

It should be understood that in general, DBD plasma can be generated with solid or hollow microneedles (e.g., FIGS. 2, 3, 5, and 6) when at least an exterior surface of the microneedle is coated with or otherwise comprises a dielectric material. When hollow, the microneedles may deliver a source gas for the plasma, a bioactive substance fluid, a cosmetic substance fluid, or combinations or mixtures thereof through the channel of the hollow microneedle. The DBD plasma jet may form when the source gas is fed through hollow microneedles as shown in FIGS. 5 and 6. For example, the DBD plasma jet may be generated as shown in FIGS. 5 and 6 by driving a source gas (represented by “F”) through channels of a hollow microneedle with the dielectric material and at least one electrode configured such that a DBD plasma jet forms and exits the tip of the microneedle (represented by “J,” FIGS. 5, 6). The source gas for such embodiments includes those described herein except for ambient air. Due to the high concentration of molecular oxygen (O₂) in ambient air, it is difficult to generate a DBD plasma jet using ambient air as the source gas. Furthermore, it is contemplated that such embodiments may generate the plasma when the microneedles of the plasmaporation device of, for example, FIGS. 5, 6 are in contact with, are penetrating the tissue or target organs of the subject, or are embedded in the tissue or target area organs of the subject, because a source gas necessary for the plasma may travel down the channel of the hollow microneedles to thereby generate the plasma. When the microneedles are solid, in certain embodiments, such as, for example, the embodiment shown in FIGS. 4, 9, the plasmaporation device needs to be spaced away from the subject so as to permit a source gas, e.g., ambient air, for the plasma to occupy or be fed to the space proximate or adjacent to the microneedles so as to generate the plasma.

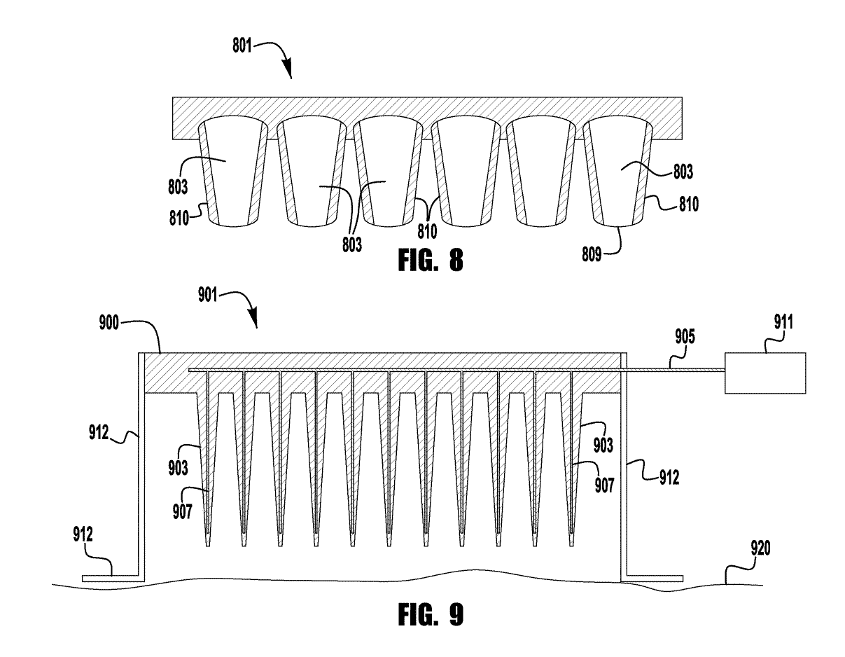

In certain embodiments, when the microneedles 803 or microneedle array comprise a dielectric material exterior surface, at least one of the electrodes optionally includes electrically grounded metallic mesh encasing the microneedle as shown in FIG. 8. In certain embodiments, the wire mesh may be spaced proximal to the exterior surface of the microneedle so that a source gas is positioned between the mesh and the microneedle. In other embodiments, the mesh contacts the exterior surface of the microneedles. When energized, this configuration generates an “indirect” DBD plasma where only neutral species pass through the mesh as the charged particles get screened by the grounded mesh. The microneedles in this embodiment may be hollow or solid.

In accordance with the embodiments of the present disclosure, the non-thermal plasma applicator energizes by a pulse having a voltage of about 1 kV to about 30 kV and a pulse duration ranging from about 1 ns to about 10,000 ns (10 μs). In addition, in accordance with the embodiments disclosed herein, non-thermal plasma applicator operates at a certain frequency or for a certain specified number of applied pulses. In accordance with exemplary embodiments, the non-thermal plasma applicator operates at a frequency of about 1 Hertz (Hz) to about 30,000 Hz (30 kHz). In accordance with certain embodiments, non-thermal plasma applicator operates with a certain selected frequency and certain selected pulse duration for a therapeutically effective amount of time. The therapeutically effective amount of time may be up to about 180 seconds, including a range from about 2 seconds to about 180 seconds, including about 2 seconds to about 60 seconds, but may vary depending on the type of substance (e.g., type of bioactive or cosmetic substance), molecular weight or size of the substance, the type of plasma being generated, and the dose of plasma being applied to the subject. In accordance with exemplary embodiments, the non-thermal plasma applicator operates at about 1 Hz to about 30,000 Hz (30 kHz) for a therapeutically effective amount of time.

As mentioned above, in certain embodiments, the non-thermal plasma applicator operates for a certain specified number of applied pulses. In accordance with exemplary embodiments, the non-thermal plasma applicator operates at from about 1 pulse to about 100,000 pulses, including from about 1 pulse to about 10,000 pulses, including from about 1 pulse to about 1,000 pulses, and including from about 1 pulse to about 500 pulses. The pulses may be triggered manually or may be automated (e.g., using an external trigger) to select the desired specified number of pulses.

In accordance with certain embodiments, the duty cycle of the non-thermal plasma applicator can be varied from about 1% to about 100%. The operational energy, pulse duration, and frequency or number of applied pulses of the non-thermal plasma applicator may differ depending on the type of plasma being generated, e.g., DBD plasma, DBD plasma jet, or a corona discharge plasma.

For example, when the plasmaporation device is a DBD plasma generator or a DBD plasma jet plasma generator, the non-thermal plasma applicator energizes by 1 kV to 30 kV pulses at a frequency of 50 Hz to about 30,000 Hz (30 kHz) for up to about 180 s, including from about 2 s to about 180 s, or from about 1 pulse to about 100,000 pulses to generate the DBD plasma or DBD plasma jet. In certain embodiments, the non-thermal plasma applicator energizes by 1 kV to 30 kV pulses at a frequency of 50 Hz to about 30,000 Hz (30 kHz) for a therapeutically effective amount of time to generate the DBD plasma or DBD plasma jet. The pulse duration for such DBD plasma or DBD plasma jet generator ranges from about 1 ns to about 10,000 ns (10 μs). Operating at a higher voltage or longer pulse duration with a DBD plasma or DBD plasma jet may result in an unpleasant effect on the subject. Additional exemplary settings for DBD plasma may be found in co-pending application Ser. No. 15/012,304, which is titled Boosting The Efficacy of DNA-Based Vaccines With Non-Thermal DBD Plasma and was filed on Feb. 1, 2016, which is incorporated herein in its entirety by reference.

When the plasmaporation device is a corona discharge plasma generator, the non-thermal plasma applicator energizes by 1 kV to 15 kV pulses at a frequency of 1 Hz to 500 Hz for up to 60 s, including from about 2 s to about 60 s, or from about 1 pulse to about 100,000 pulses to generate the corona discharge plasma. In certain embodiments, the non-thermal plasma applicator energizes by 1 kV to 15 kV pulses at a frequency of 1 Hz to 500 Hz for a therapeutically effective amount of time to generate the corona discharge plasma. The pulse duration for such corona discharge plasma generator ranges from about 1 ns to about 40 ns. Operating at a higher voltage or longer pulse duration with a corona discharge plasma may result in an unpleasant effect on the subject.

As mentioned above, in accordance with certain embodiments disclosed herein, the non-thermal plasma applicator comprises a high voltage generator. Non-limiting examples of suitable high voltage generators include a direct current (DC) power source, an alternating current (AC) power source, and a radio-frequency (RF) power source. Exemplary non-limiting DC power sources include a pulsed direct current (DC) power source. Exemplary non-limiting AC power sources include a pulsed alternating current (AC) power source. More specific examples of such high voltage generators include, but are not limited to a picosecond pulse generator, a nanosecond pulse generator, a microsecond pulse generator, or a sinusoidal generator.

### Source Gas

The plasmaporation devices use a source gas to generate the plasma. In accordance with embodiments disclosed herein, the source gas is selected from a noble gas, a molecular gas, or ambient air. As discussed above, in exemplary embodiments using a source gas and a carrier fluid, the source gas may be the same or different than the carrier fluid. In accordance with the embodiments disclosed herein, examples of suitable source gases used to generate the plasma include, but are not limited to, ambient air; noble gases such as He, Ar, Ne, Xe, and the like; molecular gases such as molecular nitrogen (N₂); a mixture of any of He, Ar, Ne, Xe, and N₂ with molecular oxygen (O₂) where the O₂ comprises less than 1% of the mixture (based on the total volume of source gas); and combinations thereof. A noble or molecular N₂ gas may be provided or supplied in neat or purified form to the plasmaporation device for use in generating the plasma.

As mentioned above, in certain embodiments, the source gas may pass through the reservoir as shown in FIGS. 5 and 6. In certain such embodiments, the source gas is in fluid communication with the channel of hollow microneedles and may be driven through or delivered through the channel to provide the necessary source of gas for plasma generation. In other embodiments, the source gas may be provided to the plasmaporation device via a separate line or feed of the source gas, e.g., not in fluid communication with the reservoir. In such embodiments, the plasmaporation device is in fluid communication with a source gas used to generate plasma although the source gas does not pass through the reservoir. In accordance with certain of the foregoing embodiments, for example, when the source gas passes through the reservoir or is fed with a separate line of feed of source gas that is not in fluid communication with the reservoir but is in fluid communication with the plasmaporation device in a manner such that plasma is generated from the source gas, the source gas is ambient air or selected from the group consisting of He; Ar; Ne; Xe; N₂; a mixture of any of He, Ar, Ne, Xe, and N₂ with O₂ where the O₂ comprises less than 1% of the mixture (based on the total volume of the source gas); and combinations thereof.

As discussed above, in certain embodiments disclosed herein, the source gas may mix with the substance being delivered. In such embodiments, as discussed above, particularly when the bioactive substance is susceptible to modification by the plasma being generated, the substance is preferably protected from the plasma generated from the source gas in some manner. In certain such embodiments for example, the substance may be encapsulated with a nanoparticle, liposome, or other protective materials known in the art.

In certain embodiments, the source gas is ambient air positioned adjacent to the plasmaporation device. In accordance with such embodiments, the source gas is the ambient air located in a gap between the plasmaporation device and the target area (cells or tissue) of the subject.

### Bioactive Substance

The plasmaporation device and methods of the present disclosure provide efficient intercellular, intracellular, or both intercellular and intracellular delivery and uptake of substances, including for example, bioactive substances by creating temporary pores in the target area (cells or tissue) of a subject using plasma. A bioactive substance as discussed herein refers to a fluid, i.e., gas or liquid, comprising molecules, drugs, vaccines, biologics, such as monoclonal antibodies or other proteins and peptides, and the like that may be delivered intercellularly, intracellularly, or both intercellularly and intracellularly to the subject. Non-limiting examples of such bioactive substances include antifungals, antimicrobials, opioids, growth factors, polynucleotides, oligonucleotides, peptides, RNAs, DNA plasmids, DNA based vaccines, RNA based vaccines, protein based vaccines, nanoparticles, micelles, vesicles, quantum dots, cytokines, chemokines, antibodies, liposomes, and drugs including, but not limited to, nonsteroidal anti-inflammatory drugs (NSAID's).

A benefit of utilizing plasmaporation in combination with the microneedles in accordance with the embodiments described herein includes allowing for the delivery of larger molecules intercellularly and/or intracellularly because of pores created or modified by the plasmaporation device disclosed herein, including pores created by the physical insertion of the microneedles into the target area and the pores created and/or modified by the plasma generated by the plasmaporation device. With respect to transdermal delivery of molecules, it should be understood that only a small percentage of compounds can be delivered transdermally because skin has significant barrier properties, namely the highly lipophilic exterior stratum corneum layer, which prevents molecules from penetrating or diffusing across the skin. As a result, only relatively small molecules, e.g., those with a molecular weight of less than 500 Da, can be administered percutaneously without the benefit of plasmaporation according to the present disclosure. Thus, when transdermal therapy or vaccination, e.g., topical dermatological therapy, percutaneous systemic therapy, or vaccination is the objective, the development of innovative compounds for pharmaceutical applications is restricted to a molecular weight of less than, for example, 500 Dalton. In addition, transport of most drugs across the skin is very slow, and lag times to reach steady-state fluxes are measured in hours. Achievement of a therapeutically effective drug level is therefore difficult without artificially enhancing skin permeation. Applicants have developed techniques for moving molecules and DNA across layers of the skin, both intracellularly (into the cells) and intercellularly (between the cells) using plasma. Applicant filed U.S. patent application Ser. No. 14/500,144 entitled Method and Apparatus for Delivery of Molecules across Layers of the Skin on Sep. 29, 2014, which is incorporated herein by reference in its entirety. In this case, Applicant shows exemplary methods utilizing non-thermal plasma for providing a safe, contactless transdermal delivery and cellular uptake of DNA vaccines via plasmaporation.

It should be understood that according to certain embodiments disclosed herein, the bioactive substance may be exposed to the plasma generated by the plasmaporation devices disclosed herein. For example, if the source gas used to generate plasma mixes with the bioactive substance, as discussed above, the plasma may interact and modify or change the bioactive substance. Alternatively, if the bioactive substance is applied to the target area before the plasma, the bioactive substance may be susceptible to change or modification upon exposure to the plasma. In such embodiments, particularly when the bioactive substance is susceptible to being changed or modified, the bioactive substance is preferably protected from the plasma generated from the source gas in some manner. In certain such embodiments for example, the bioactive substance may be encapsulated with a nanoparticle, liposome, or other protective materials known in the art.

In accordance with embodiments disclosed herein, the bioactive substance has a molecular weight up to about 5,000,000 Daltons (Da, which is about 5,000 kDa), including from about 500 Da to about 5,000,000 Da. In certain embodiments, the bioactive substance has a molecular weight up to about 150,000 Da (about 150 kDa), including from about 500 Da to about 150,000 Da. Accordingly, in certain other embodiments, the bioactive substance has a molecular weight of about 150,000 Da to about 5,000,000 Da. Such benefit in promoting the delivery of larger molecules may also be characterized by the size of the bioactive substance. In accordance with certain embodiments disclosed herein, the bioactive substance has a size of about 0.02 μm (20 nm) to about 50 μm. Unless otherwise indicated, the term “size” in the context of the bioactive substance or cosmetic substance refers to the longest length dimension or diameter of the substance.

Exemplary bioactive substances, cosmetics and other substances that may be used with the present invention are disclosed in U.S. Pat. Pub. No. 2015/0094647 titled METHODS AND APPARATUS FOR DELIVERY OF MOLECULES ACROSS LAYERS OF TISSUE filed on Sep. 29, 2014, which is incorporated herein in its entirety for its disclosure on exemplary bioactive substances, cosmetics and other substances that may be used with the present invention.

### Cosmetic Substance

The plasmaporation device and methods of the present disclosure also provide efficient intercellular, intracellular, or both intercellular and intracellular delivery and uptake of fluids, i.e., gases or liquids, comprising cosmetic substances by creating temporary pores in the target area (cells or tissue) of a subject using plasma. Non-limiting examples of such cosmetic substances include botulinum toxin A or B (Botox), hyaluronic acid, collagen, moisturizers, growth factors, antiwrinkle creams, emollients, ointments, and the like. In some embodiments, cosmetice include chemical enhancers, such as, dimethyl sulfoxide, azone, pyrrolidones, oxazolidinones, urea, oleic acid, ethanol, liposomes and the like.

It should be understood that according to certain embodiments disclosed herein, the cosmetic substance may be exposed to the plasma generated by the plasmaporation devices disclosed herein. For example, if the source gas used to generate plasma mixes with the cosmetic substance, as discussed above, the plasma may interact and modify or change the cosmetic substance. Alternatively, if the cosmetic substance is applied to the target area before the plasma, the cosmetic substance may be susceptible to change or modification upon exposure to the plasma. In such embodiments, particularly when the cosmetic substance is susceptible to being changed or modified, the cosmetic substance is preferably protected from the plasma generated from the source gas in some manner. In certain such embodiments for example, the cosmetic substance may be encapsulated in a nanoparticle, liposome, or other protective materials known in the art.

In accordance with embodiments disclosed herein, the cosmetic substance has a molecular weight up to about 5,000,000 Da, including from about 500 Da to about 5,000,000 Da. In certain embodiments, the cosmetic substance has a molecular weight up to about 150,000 Da, including from about 500 Da to about 150,000 Da. Accordingly, in certain other embodiments, the cosmetic substance has a molecular weight of about 150,000 Da to about 5,000,000 Da. The cosmetic substance may also be characterized by the size of the cosmetic substance. In accordance with certain embodiments disclosed herein, the cosmetic substance has a size of about 0.02 μm (20 nm) to about 50 μm.

As stated above, exemplary bioactive substances, cosmetics and other substances that may be used with the present invention are disclosed in U.S. Pat. Pub. No. 2015/0094647 titled METHODS AND APPARATUS FOR DELIVERY OF MOLECULES ACROSS LAYERS OF TISSUE filed on Sep. 29, 2014, which is incorporated herein in its entirety for its disclosure on exemplary bioactive substances, cosmetics and other substances that may be used with the present invention.

### Exemplary Embodiments

FIGS. 2-8 show exemplary plasmaporation devices in accordance with the present disclosure.

FIG. 2 shows an exemplary plasmaporation device 201 containing hollow microneedles 203. In accordance with this exemplary embodiment, the microneedles 203 and microneedle array 200 are made with a dielectric material. The dielectric material may include any such dielectric materials disclosed herein. Electrode 207 is embedded in the microneedles 203 and microneedle array 200 and is in circuit communication to a high voltage generator 211 via conductor 205. In accordance with this embodiment, the non-thermal plasma applicator comprises electrode 207 in circuit communication with high voltage generator 211. The electrode 207 is a conductive material such as the metals disclosed herein.

The plasmaporation device 201 includes a reservoir 204, which may contain a substance such as a bioactive substance or a cosmetic substance, a carrier fluid, and combinations thereof in accordance with the embodiments of the present disclosure. The substance travels through channels 206 of the hollow microneedle 203 and out the tip 209 of the hollow microneedle when applied to the subject.

Because the electrode 207 is embedded in the dielectric (insulating) material of the microneedles 203/microneedle array 200, the plasma generated may be a DBD plasma or DBD plasma jet. The plasma generated by this embodiment will be a DBD plasma if the source gas is ambient air surrounding the exterior surface of the microneedles 203, and a DBD plasma jet if source gas flows through the microneedles 203.

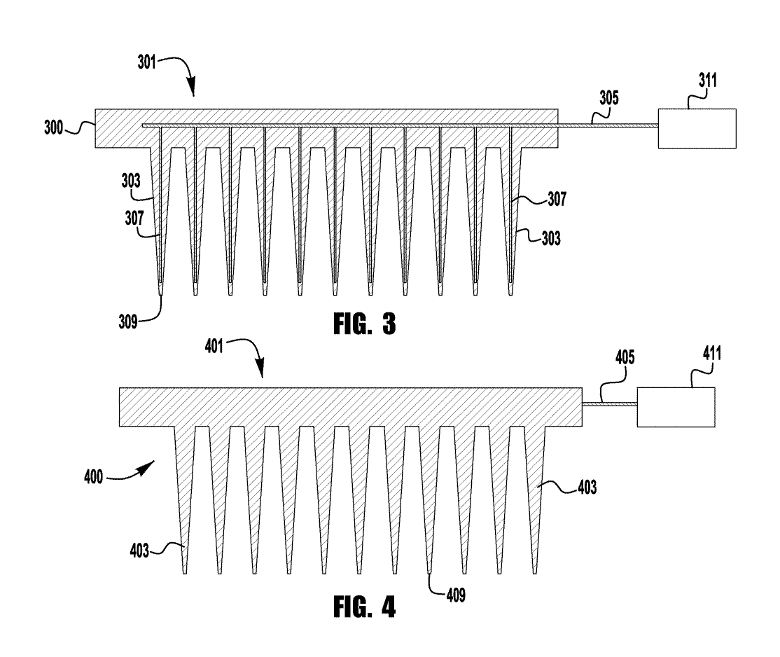

FIG. 3 shows an exemplary plasmaporation device 301 containing solid microneedles 303. In accordance with this exemplary embodiment, the microneedles 303 and microneedle array 300 are made of a dielectric material. The dielectric material may include any such dielectric materials disclosed herein. Electrode 307 is embedded in the dielectric microneedles 303 and microneedle array 300 and is in circuit communication to a high voltage generator 311 via high voltage conductor 305. In accordance with this embodiment, the non-thermal plasma applicator comprises electrode 307 in circuit communication with high voltage generator 311. The electrode 307 is a conductive material such as the metals disclosed herein.

Because the electrode 307 is embedded in the dielectric (insulated) material (300 and 303), in some embodiments, the plasma generated will be a DBD plasma using ambient air surrounding the exterior surface of the microneedles 303 as the source gas for the plasma.

In accordance with this embodiment, the substance, such as the bioactive substance or cosmetic substance may be delivered intercellularly and/or intracellularly after the plasma generated by this plasmaporation device is applied to the subject. This may be accomplished by topically applying the substance to the target area of the skin before or after the plasma generation. Alternatively, this may be accomplished by at least partially coating the surface of the microneedles 303, including for example coating the tips 309 of the microneedles, and contacting the microneedles 303 coated with the substance with the target area of the subject before or after the generation of the plasma. In certain embodiments, contacting the microneedles 303 with the target area includes inserting the microneedles 303 into the target area tissue or cells.

FIG. 4 shows an exemplary plasmaporation device 401 containing solid microneedles 403. In accordance with this exemplary embodiment, the microneedles 403 and/or microneedle array 400 comprise a conductive material. The conductive material may include any such conductive materials disclosed herein. Because the microneedles 403 and/or microneedle array 400 are a conductive material, it is not necessary to embed separate electrodes in the microneedles 403 and/or microneedle array 400 as is done when the microneedles and microneedle array comprise dielectric (insulated) material. In accordance with this embodiment, the non-thermal plasma applicator comprises the high voltage generator 411 and microneedle array 400.

In accordance with some embodiments, the plasma generated will be a corona discharge plasma using ambient air surrounding the exterior surface of the microneedles 403 as the source gas for the plasma. In certain other embodiments, the microneedles 403 and/or microneedle array 400 may be coated with a dielectric material (not shown in FIG. 4). In such embodiments when the conductive microneedles 403 are coated with a dielectric (insulated) material, the plasma generated will be a DBD plasma using ambient air surrounding the exterior surface of the dielectric coated microneedles 403 as the source gas for the plasma.

In accordance with this exemplary embodiment, the substance, such as the bioactive substance or cosmetic substance, may be delivered intercellularly and/or intracellularly after the plasma generated by this plasmaporation device is applied to the subject. This may be accomplished by topically applying to the target area of the skin the substance before or after the plasma generation. Alternatively, this may be accomplished by at least partially coating the exterior surface of the microneedles 403, including for example coating the tips 409 of the microneedles, and contacting the microneedles 403 coated with the substance with the target area before or after the generation of the plasma. In certain embodiments, contacting the microneedles 403 with the target area includes inserting the microneedles into the target area tissue or cells.

FIGS. 5 and 6 show exemplary plasmaporation devices 501, 601 containing hollow microneedles 503. In accordance with these exemplary embodiments, the microneedles 503, 603 and microneedle arrays 500, 600 are made of a dielectric material. The dielectric material may include any such dielectric materials disclosed herein. Electrodes 507 (FIG. 5), 607 (FIG. 6) are embedded in the dielectric microneedles 503, 603 and microneedle arrays 500, 600 and is in circuit communication to a high voltage generator 511, 611 via lead 505, 605 respectively. In accordance with these embodiments, the non-thermal plasma applicators include electrodes 507. 607 in circuit communication with high voltage generator 511, 611 respectively. The difference between the respective embodiments shown in FIGS. 5 and 6 is the configuration of the electrodes 507, 607. As shown in FIG. 5, the electrode 507 extends substantially along the axial length (distance “h”) of the microneedle 503. In contrast, the electrode 607 in FIG. 6 is embedded at the base of the microneedle 603 (opposite the axial length from the tip 609), thus requiring the source gas for the plasma to pass through channel 606 and passed electrode 607 to generate a DBD plasma jet.

The plasmaporation device includes a reservoirs 504, 604, which may contain a substance such as a bioactive substance or a cosmetic substance, a carrier fluid, and combinations thereof in accordance with the embodiments of the present disclosure. The reservoirs 404, 604 are also in fluid communication with a source of the source gas intended to be driven through the hollow microneedle as shown by the flow “F” of fluid into reservoirs 504, 604. When a combination of the substance in the reservoir, the source gas, and optionally the carrier fluid are utilized with this embodiment, the fluids may be provided in serial and delivered through the hollow microneedles in serial. Alternatively, when a combination of the substance in the reservoir, the source gas, and optionally the carrier fluid are utilized, the combination may be a mixture of one or more of the source gas, the substance such as bioactive substance or cosmetic substance, and the carrier fluid. Such mixture may be delivered through the hollow microneedles as a mixture (i.e., not in serial). In such embodiments, the substance may be protected as disclosed herein to prevent changes or modifications that may occur by the bioactive substance being exposed to the plasma.

The substance travels through channels 506, 606 of the hollow microneedles 503, 603 respectively and out the tips 509, 609 of the hollow microneedles when applied to the subject. When the source gas is driven through channel 506, 606, it exits out of tip 509, 609 to generate a DBD plasma jet (shown as “J” in FIGS. 5 and 6).

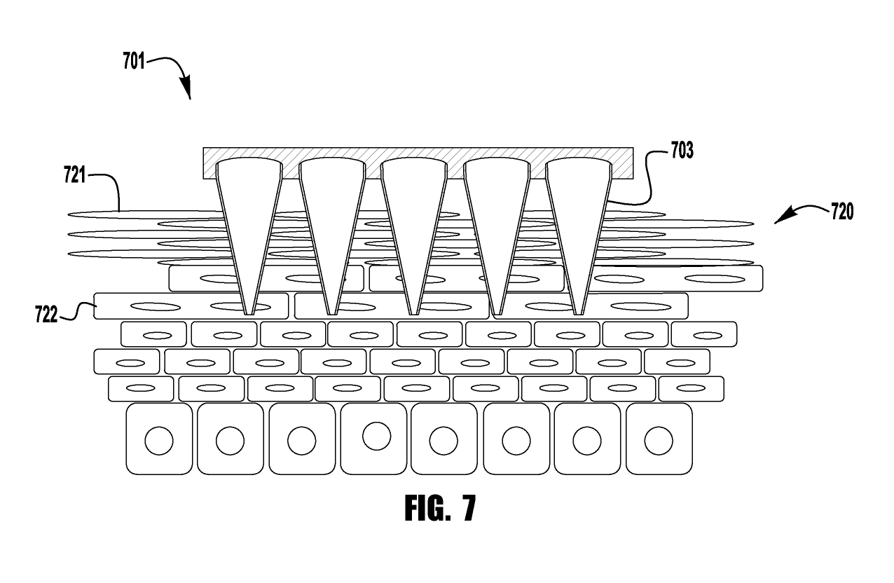

FIG. 7 is a cross-sectional elevational view illustrating the use of a portion of a plasmaporation device 701 disclosed herein where the microneedles 703 have penetrated the epidermis 720 of the skin of the subject. The microneedles 703 of FIG. 7 is intended to represent the penetration of the skin to a depth of about 60 μm to about 200 μm, where the tip of the microneedles have bypassed the highly lipophilic stratum corneum layer of the skin 721 and are positioned in the stratum granulosum layer 722. However, as discussed above, the height of microneedle 703 could be much larger, e.g., up to 2,000 μm, and therefore the penetration depth can be larger than that about 150 μm to about 300 μm represented in FIG. 7.

FIG. 8 shows a portion of an exemplary plasmaporation device 801 containing solid microneedles 803 or microneedle array 800 comprise a dielectric material exterior surface, at least one of the electrodes includes electrically grounded metallic mesh 810 encasing the microneedle 803. In certain embodiments, the wire mesh 810 may be spaced proximal to the exterior surface of the microneedle 803 so that a source gas is positioned between the mesh 810 and the microneedle 803. In other embodiments, the mesh 810 contacts the exterior surface of the microneedles 803. When energized, this configuration generates an “indirect” DBD plasma where only neutral species pass through the mesh 810 as the charged particles get screened by the grounded mesh. The plasmaporation device 801 according to this embodiment thus generates a DBD plasma using ambient air as the source gas for the plasma. In some embodiments, the microneedles of this embodiment may be hollow microneedles in fluid communication with a reservoir (not shown). The reservoir in such embodiment may contain a substance, such as a bioactive substance or a cosmetic substance, a carrier gas for such substance, or a mixture of both for delivery to the subject.

FIG. 9 shows a cross-sectional elevation view of an exemplary plasmaporation device 901 having solid microneedles 903 spaced from the target area 920 of the subject using a spacer component 912. In certain embodiments disclosed herein, the plasmaporation device may optionally include a spacer component 912 that extends from the plasmaporation device to the subject for use when generating the plasma. A non-limiting exemplary spacer component 912 is shown in FIG. 9. When extended, the spacer component 912 prevents the microneedles and/or microneedle array, from physically contacting the subject. For example, as shown in FIG. 9, the tips 909 of the microneedles 903 are proximate to, but not in contact with, the target area 920 of the subject. The spacer component can be used with any plasmaporation device disclosed herein, including those plasmaporation devices 201 with hollow microneedles 203 (not shown) and those plasmaporation devices 101 having solid microneedles 103. The spacer component is useful for several different functions, including but not limited to, providing space for the source gas used to generate the plasma, providing the an optimal distance between the plasmaporation device and the subject for application of the plasma to the subject, and preventing shocking in certain applications, however, it is preferable to operate the plasmaporation device in a mode that prevents shocking, such as, for example, operating at low pulse durations, e.g. in the nanosecond regime, which would not shock a subject even if the microneedles contacted the subject. With respect to the space function, certain embodiments of the plasmaporation device disclosed herein, such as ones with solid microneedles or hollow microneedles in which the source gas is not fed through a channel of the microneedle, need a source of gas to be present between the device and the subject. The spacer component provides a gap between the plasmaporation device and the target area of the subject as is necessary to generate the plasma from the source gas (e.g., ambient air) present in the gap. In some embodiments, the gap provided by the spacer component is from about 1 mm to about 3 mm. In other words, the gap shown between the tips 909 of microneedles 903 the target area 920 of the subject in FIG. 9 is from about 1 mm to about 3 mm. After the plasma is generated and applied to the subject, the spacer component can be repositioned or removed from the plasmaporation device so that the microneedle portion of the device can be contacted to the target area of the subject. One of ordinary skill in the art would understand how to incorporate a spacer component so that it does not interfere with the plasma being applied to the target area, as well as how it may be repositioned or removed such that the microneedles can contact or penetrate the target area of the subject.

In accordance with some embodiments, the spacer component comprises a non-conductive material. Non-limiting examples of such suitable materials for the spacer component include polymeric materials such as synthetic or natural rubbers; silicone; neoprene; PTFE; PEI; polystyrene foam; plastics such as polycarbonate, polyethylene, polyurethane; and the like.

In accordance with other embodiments, as discussed above, the spacer component comprises a grounding conductor to provide a ground path between the target area of the subject back to the power supply (not shown). In such embodiments, the grounding conductor comprises any of the conductive materials disclosed herein.

### Method of Use

In the plasma phase, neutral gas atoms (or molecules), electrons, positive/negative ions, and radicals are generated. Their generation and concentration depend, in part, on the physical and chemical properties of the gas being used to generate the plasma as well as the electrical parameters used to generate the plasma. The strength of the electric field generated by non-thermal plasma on skin can be tuned by varying the composition of the source gas, the time of plasma treatment, gap distance between the electrode and the skin, applied voltage, pulse duration, frequency, and duty cycle to localize delivery. These parameters allow control of the depth and delivery amount of substance, e.g., bioactive or cosmetic substance, and other substances disclosed herein or incorporated herein into the target area. Thus, depending on the plasma dose, the depth of penetration of the substance can be regulated to ensure delivery to the target layer (e.g., stratum corneum, epidermis and dermis) of the target area of the subject.

Other embodiments of the present disclosure include methods for delivering substances, such as bioactive substances, cosmetic substances, and other substances disclosed herein or incorporated herein using plasmaporation and microneedles. In certain such embodiments, the methods include using the plasmaporation devices disclosed herein to deliver a substance, such as a bioactive substance or a cosmetic substance, to a target area of the subject. In accordance with certain embodiments disclosed herein, the substance is delivered via the microneedles, such as being delivered through the hollow microneedles or by being coated on the solid microneedles so that when the microneedles contact the target area of the subject and the cells or tissue of the target area of the subject has been porated by the plasma, the substance is delivered at least one of intercellularly and intracellularly to the cells or tissue of the target area. As discussed in greater detail below, when the microneedles contact the target area of the subject, the microneedles may penetrate the tissue of the target area. In certain embodiments, the substance is further delivered to the bloodstream of the subject.

Examples of suitable target areas include any human or animal organ containing cells or tissue capable of the creation of pores intercellularly, intracellularly, or both intercellularly and intracellularly. In accordance with certain preferred embodiments disclosed herein, the target area is exposed to the environment. Specific non-limiting examples of such target areas include the skin, eyes, sublingual mucosal membrane, buccal mucosal membrane, nasal mucosal membrane, nails, and the like. In accordance with other embodiments, the target area is internal to the subject, such as the heart, lungs, stomach, pancreas, liver, kidneys, and any other internal organs, such that the plasmaporation device is applied invasively by being implanted or via open surgery.

The delivery of the substance in such embodiments to the target area may take place before or after the application of the plasma to the cells or tissue of the subject. In accordance with certain other embodiments disclosed herein, the substance is delivered via the direct topical application of the substance to the cells or tissue of the subject. In such embodiments, the plasma is applied before or after the direct topical application of the substance to the cells or tissue of the subject. Several exemplary methods for delivering a substance in accordance with the embodiments of the present disclosure are provided below.

In accordance with certain embodiments, a method for delivery of a substance, such as a bioactive or cosmetic substance, includes using a plasmaporation device to deliver a substance the cells or tissue of a target area of a subject. In accordance with this exemplary embodiment, the plasmaporation device comprises hollow microneedles, a non-thermal plasma generator, and a reservoir containing a substance in fluid communication with the hollow microneedles. Power is then provided to generate a plasma by a generator delivering one or more pulses having a voltage of about 1 kV to about 30 kV and a pulse duration ranging from about 1 ns to about 10,000 ns, the non-thermal plasma generator operates at (i) a pulse frequency of about 1 Hz to about 30,000 Hz for up to about 180 s or (ii) from about 1 to about 100,000 pulses, wherein the plasma porates the cells or tissue of the target area. Next, the substance, such as a bioactive substance or cosmetic substance, is delivered from the reservoir through the hollow microneedles into the porated cells or tissue of the target area of the subject. In certain embodiments in accordance with this method, the plasmaporation device applies plasma to the target area for 2 s to 180 s, including from about 2 s to about 60 s. Thus, in accordance with such embodiments, for example when a voltage, pulse duration, and frequency is selected, power may be provided to the plasmaporation device to generate a plasma for up to 180 s, including from about 2 s to about 180 s, including from about 2 s to about 60 s.

As used herein, the terms “power may be provided to the plasma poration device” for a specified period of time does not necessarily mean that continuous power is applied to the plasma poration device. For example, the power may be a series of pulses provided to the plasma poration device over a specified period of time.

Unless otherwise indicated herein, the aspect “applying a plasmaporation device to skin, tissue or cells of a target area of a subject” means positioning or placing the plasmaporation device proximal to, or in contact with, the target area of the subject such that the plasmaporation device is positioned to provide a dose of plasma sufficient to create temporary pores in the tissue or cells to effect the delivery of the substance.

Further in accordance with some embodiments, the step in which the substance is delivered from the reservoir includes contacting the microneedles to the target area of the subject if the plasmaporation device is not already in contact with the target area. While in contact with the target area, the substance is delivered or otherwise transferred from the plasmaporation device to the cells, tissue, or bloodstream of the subject. In certain embodiments, contacting the microneedles with the target area includes inserting the microneedles into the target area tissue or cells.

In accordance with some embodiments, a method for delivery of a substance, such as a bioactive or cosmetic substance, includes using a plasmaporation device to deliver a substance to cells or tissue of a target area of a subject. The plasmaporation device comprises hollow microneedles, a non-thermal plasma generator, and a reservoir containing a substance in fluid communication with the hollow microneedles. The substance is delivered from the reservoir through the hollow microneedles into the cells or tissue of the target area of the subject, and power is provided by one or more pulses having a voltage of about 1 kV to about 30 kV and a pulse duration ranging from about 1 ns to about 10,000 ns, the non-thermal plasma generator operates at (i) a pulse frequency of about 1 Hz to about 30,000 Hz for up to about 180 s or (ii) from about 1 to about 100,000 pulses, wherein the plasma porates the cells or tissue of the target area to facilitate transfer of the substance into the porated cells or tissue of the target area of the subject. In accordance with this embodiment, the step in which the substance is delivered from the reservoir further includes contacting the microneedles to the target area of the subject if the plasmaporation device is not already in contact with the target area. In certain embodiments, contacting the microneedles with the target area includes inserting the microneedles into the target area tissue or cells. While in contact with the target area, the substance is delivered or otherwise transferred from the plasmaporation device to the cells or tissue. In certain embodiments in accordance with this method, the plasmaporation device applies plasma to the target area for 2 s to 180 s, including from about 2 s to about 60 s. Thus, in accordance with such embodiments, for example when a voltage, pulse duration, and frequency is selected, power may be provided to the plasmaporation device to generate a plasma for up to 180 s, including from about 2 s to about 180 s, including from about 2 s to about 60 s.

In accordance with some embodiments, a method for delivery of a substance, such as a bioactive or cosmetic substance, includes using a plasmaporation device to deliver a substance to cells or tissue of a target area of a subject, wherein the plasmaporation device comprises solid microneedles and a non-thermal plasma generator, and wherein the solid microneedles are at least partially coated with a substance for delivery through the target area. The substance is delivered by contacting the target area of the subject with the solid microneedles at least partially coated with the substance. Power is provided to the plasmaporation device to generate a plasma by energizing the non-thermal plasma generator and delivering a pulse having a voltage of about 1 kV to about 30 kV and a pulse duration ranging from about 1 ns to about 10,000 ns, the non-thermal plasma applicator operates at (i) a pulse frequency of about 1 Hz to about 30,000 Hz for up to about 180 s or (ii) from about 1 to about 100,000 pulses. In accordance with this embodiment, the plasma porates the cells or tissue of the target area to transfer or at least facilitate transfer of the substance to the subject. The delivery of the substance occurs before or after applying power to the plasmaporation device to generate the plasma. In certain embodiments in accordance with this method, the plasmaporation device applies plasma to the target area for 2 s to 180 s, including from about 2 s to about 60 s. Thus, in accordance with such embodiments, for example when a voltage, pulse duration, and frequency is selected, power may be provided to the plasmaporation device to generate a plasma for up to 180 s, including from about 2 s to about 180 s, including from about 2 s to about 60 s.

In accordance with some embodiments, a method for delivery of a substance, such as a bioactive substance or a cosmetic substance, includes using a plasmaporation device to deliver a substance to the cells or tissue of a target area of a subject, where the plasmaporation device comprises solid or hollow microneedles and a non-thermal plasma applicator. In accordance with this embodiment, power is provided to generate a plasma to thereby create or modify pores by energizing the non-thermal plasma applicator. The power signal is one or more pulses having a voltage of about 1 kV to about 30 kV and a pulse duration ranging from about 1 ns to about 10,000 ns, the non-thermal plasma applicator operates at (i) a pulse frequency of about 1 Hz to about 30,000 Hz for up to about 180 s or (ii) from about 1 to about 100,000 pulses. The substance is topically applied (i.e., contacted) to the cells or tissue of the target area of the subject containing the pores created or modified by the plasmaporation device. The substance may be topically applied before or after the pores are created or modified. In certain embodiments in accordance with this method, the plasmaporation device applies plasma to the target area for 2 s to 180 s, including from about 2 s to about 60 s. Thus, in accordance with such embodiments, for example when a voltage, pulse duration, and frequency is selected, power may be provided to the plasmaporation device to generate a plasma for up to 180 s, including from about 2 s to about 180 s, including from about 2 s to about 60 s.

In accordance with the preceding embodiment, when the substance is topically applied to the cells after the pores are created or modified by the plasma generated by the plasmaporation device, the method may further comprise contacting the microneedles to the target area so as to penetrate the tissue of the target area to a certain desired depth; and providing power to the plasmaporation device to generate a plasma to facilitate intracellular delivery of the substance previously topically applied. Prior to contacting the microneedles in accordance with this embodiment, the method may further include waiting for a period of time, including for example a predetermined amount of time, prior to contacting the microneedles to the target area so as to penetrate the target area to a predetermined depth.

In accordance with the certain embodiments of the methods disclosed herein, the microneedles may penetrate the target area up to a depth of 300 μm when contacted to the target area. In accordance with the certain embodiments of the methods disclosed herein, the microneedles may penetrate tissue, e.g., at least one of skin, epithelial tissue, mucosal tissue, connective tissue, muscle tissue, and nervous tissue, up to a depth of 300 μm when contacted to the target area. In other embodiments, the microneedles do not penetrate the target area of the subject when contacted to the target area. In certain embodiments, the microneedles do not penetrate tissue or cells of the subject when contacted to the target area.

The operational parameters including the microneedle material of the plasmaporation device of the foregoing exemplary methods are in accordance with those parameters disclosed herein (as it should be understood that the operational parameters and the microneedle material differ for DBD plasma or DBD plasma jet generators versus a corona discharge plasma generators).

Embodiments that disclose use of the present invention with bioactive substances or cosmetics are meant to include other substances and are not limited to these identified categories and may include other substances, such as, for example, those disclosed herein or incorporated herein

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described. Accordingly, departures may be made from such details without departing from the spirit or scope of the applicant's general disclosure herein.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

As used in the description of the invention and the appended claims, the singular forms “a,” “an,” and “the” are intended to include the plural forms as well, unless the context clearly indicates otherwise.

What is claimed is:

1. A plasmaporation device comprising,
microneedles, and
a non-thermal plasma applicator, wherein the non-thermal plasma applicator energizes an electrode with one or more pulses having a voltage of about 1 kV to about 30 kV and a pulse duration ranging from about 1 ns to about 10,000 ns, and wherein the non-thermal plasma applicator operates at (i) a pulse frequency of about 1 Hz to about 30,000 Hz for up to about 180 s or (ii) from about 1 to about 100,000 pulses.

2. The plasmaporation device of claim 1, wherein the non-thermal plasma generator includes at least one high voltage generator selected from a DC power source, an AC power source, and a RF power source.

3. The plasmaporation device of claim 1, wherein the non-thermal plasma generator includes at least one high voltage generator and the at least one high voltage generator is a picosecond pulse generator, a nanosecond pulse generator, a microsecond pulse generator, or a sinusoidal generator.

4. The plasmaporation device of claim 1, wherein the plasmaporation device is one of a dielectric barrier discharge (DBD) plasma generator, a DBD plasma jet plasma generator, and a corona discharge plasma generator.

5. The plasmaporation device of claim 1, wherein the microneedles comprise a conductive material.

6. The plasmaporation device of claim 1, wherein the plasmaporation device is a corona discharge plasma generator.

7. The plasmaporation device of claim 1, wherein the microneedles are an electrode and the non-thermal plasma applicator energizes the microneedles with pulses that have a pulse duration ranging from about 1 ns to about 40 ns.

8. The plasmaporation device of claim 1, wherein an exterior surface of the microneedles comprises a dielectric material.

9. The plasmaporation device of claim 8, wherein the dielectric material is selected from the group consisting of a polytetrafluoroethylene, aluminum oxide, a silicone, natural rubber, a synthetic rubber, ceramic, polyetherimide, quartz, and magnesium fluoride.

10. The plasmaporation device of claim 1, wherein the plasmaporation device generates a DBD plasma or a DBD plasma jet.

11. The plasmaporation device of claim 1, wherein the device further comprises an electrically grounded metallic mesh encasing the microneedles.

12. The plasmaporation device of claim 1, wherein the at least a portion of the microneedles are hollow.

13. The plasmaporation device of claim 12, wherein the plasmaporation device further comprises a reservoir in fluid communication with at least a portion of the hollow microneedles such that a substance may travel from the reservoir through the hollow microneedle and exit out of the tip of the microneedle.

14. The plasmaporation device of claim 1, wherein the plasmaporation device is in fluid communication with a source gas used to generate plasma.

15. The plasmaporation device of claim 1, wherein the substance includes at least one bioactive substance.

16. The plasmaporation device of claim 1, wherein the substance includes a cosmetic sub stance.

17. A method for delivery of a substance, the method comprising:
a. applying a plasmaporation device to cells or tissue of a target area of a subject, wherein the plasmaporation device comprises solid or hollow microneedles and a non-thermal plasma applicator;
b. providing power to the plasmaporation device to generate a plasma to thereby create or modify pores, wherein the power is one or more pulses having a voltage of about 1 kV to about 30 kV and a pulse duration ranging from about 1 ns to about 10,000 ns, the non-thermal plasma applicator operates at (i) a pulse frequency of about 1 Hz to about 30,000 Hz for up to about 180 s or (ii) from about 1 to about 100,000 pulses; and
c. topically applying a substance to the cells or tissue of the target area of the subject containing the pores created or modified by the plasmaporation device,
wherein the substance is topically applied before or after the pores are created or modified.

18. The method of claim 17, wherein when the substance is topically applied to the cells after the pores are created or modified by the plasma generated by the plasmaporation device, the method further comprises:
contacting the microneedles to the target area so as to penetrate the tissue of the target area; and
providing power to generate a plasma to facilitate intracellular delivery of the substance previously topically applied.

19. The method of claim 17, wherein the substance includes at least one of a bioactive substance or a cosmetic substance.

20. A plasmaporation device comprising,
a plurality of hollow microneedles,
an electrode;
a reservoir in fluid communication with the hollow microneedles; and
a non-thermal plasma applicator, wherein the non-thermal plasma applicator energizes the electrode to create plasma proximate the microneedles.
